(19) United States
(12) Patent Application Publication
(10) Pub. No.: US 2008/0066549 A1
(43) Pub. Date: Mar. 20, 2008

(54) METHODS, SYSTEMS AND APPARATUS FOR LIGHT CONCENTRATING MECHANISMS

(76) Inventors: Mark F. OLDHAM, Los Gatos, CA (US); Eric S. Nordman, Palo Alto, CA (US); Charles R. Connell, Redwood City, CA (US); Timothy M. Woudenberg, Foster City, CA (US)

Correspondence Address: MH2 TECHNOLOGY LAW GROUP, LLP, 1951 KIDWELL DRIVE, SUITE 550, TYSONS CORNER, VA, 22182, US

(21) Appl. No.: 11/857,419

(22) Filed: Sep. 18, 2007

Related U.S. Application Data
(60) Provisional application No. 60/826,079, filed on Sep. 18, 2006.

(51) Int. Cl. G01N 33/00

(52) U.S. Cl. 73/579; 977/762

(57) ABSTRACT

An embodiment relates generally to a method for analysis of a nucleic acid. The method includes providing for a resonant structure configured to couple with one or more fluorescently labeled nucleic acids and directing an excitation light from a source on the resonant structure. The method also includes generating plasmons on the surface of the resonant structure where the analyte is fixed at a point of energy concentration of the resonant structure.

### FIELD

This invention relates generally to light concentrating or enhancing mechanisms, more particularly to methods, apparatus and systems for light concentrating mechanisms to create a high energy field based on surface plasmons on a peripheral resonant cavity.

### DESCRIPTION OF THE RELATED ART

In non-stepwise single molecule sequencing (either free running or utilizing photo labile blockers) using labeled nucleotides, it is necessary to effect a methodology to reduce the background from the labeled nucleotides so that the labels associated with the nucleotides that are incorporated can be properly observed. Some previously described methodologies include zero mode waveguides, plasmon resonance combined with quenching photo labile linkers, FRET pairs between the enzyme and the nucleotides, exclusion layers combined with TIRE, and similar other techniques.

The conventional methodologies have drawbacks and disadvantages. For example, a typical methodology typically involves blocking the excitation light in a small area. This excitation light typically requires large expensive laser. Moreover, this methodology may generate a considerable amount of background noise, which degrades the signal quality.

### SUMMARY

An embodiment relates generally to a method for analysis of a nucleic acid. The method includes providing for a resonant structure configured to couple with one or more fluorescently labeled nucleic acids and directing an excitation light from a source on the resonant structure. The method also includes generating plasmons on the surface of the resonant structure where the analyte is fixed at a point of energy concentration of the resonant structure.

Another embodiment generally pertains to a method for analysis of an analyte. The method includes providing for a resonant structure coupled with an analyte and directing an excitation light from a source on the resonant structure. The method also includes generating plasmons on the surface of the resonant structure, where the analyte is complexed with a molecule fixed at a point of energy concentration of the resonant structure through a photoactivatable linker.

Yet another embodiment relates generally to a plasmonic structure. The plasmonic structure includes a nano-antenna implemented with a metal material and configured to generate an enhancement zone and a blocking layer deposited adjacent to a portion of the nano-antenna. The blocking layer is configured to substantially reduce the excitation of fluorophores outside of the enhancement zone.

### BRIEF DESCRIPTION OF THE DRAWINGS

Various features of the embodiments can be more fully appreciated, as the same become better understood with reference to the following detailed description of the embodiments when considered in connection with the accompanying figures, in which:

FIG. 1 illustrates an exemplary nanorice, a type of nanoparticle, in accordance with an embodiment of the present invention;

FIG. 2 illustrates an exemplary nanocrescent in accordance with another embodiment;

FIG. 3A illustrates an intensity image of a nanocrescent;

FIG. 3B illustrates a conventional zero mode wave guide;

FIG. 3C illustrates another embodiment of a resonant structure in accordance with an embodiment;

FIG. 4 illustrates a sub-wavelength hole array in accordance with yet another embodiment;

FIG. 5 illustrates a near field scanning microscope image of an energy pattern of a subwavelength hole array;

FIG. 6 illustrates a blunt tip optical fiber in accordance with yet another embodiment;

FIG. 7 illustrates a planar photonic waveguide structure in accordance with yet another embodiment;

FIG. 8 shows an intensity profile of a planar photonic waveguide structure;

FIG. 9 illustrates an embodiment of a two dimensional photonic crystal;

FIG. 10 illustrates an exemplary nano-antenna in accordance with an embodiment;

FIG. 11 illustrates an exemplary bow-tie antenna;

FIG. 12 illustrates a series of fractal nano-antennas; and

FIGS. 13A-B illustrate a coated bow-tie antenna in accordance with yet another embodiment.

### DEFINITIONS

The following terms are used to describe the various embodiments detailed below.

Plasmon resonance can be defined as a collective oscillation of free electrons or plasmons at optical frequencies.

Surface plasmons are those plasmons that are confined to surfaces and that interact strongly with light resulting in a polariton. They occur at the interface of a material with a positive dielectric constant with that of a negative dielectric constant (usually a metal or doped dielectric).

Resonant structure can refer to a structure such as a nano-antenna or nano-particles that use plasmon resonance along with shape of the structure to concentrate light energy to create a small zone of high local field.

Fluorescence enhancement ratio PER) can refer to a ratio of the fluorescence photons collected from the excitation zone associated with a resonant structure element relative to the photons that would be collected from an equivalent sized zone with no resonant structure element and with all other variables held constant.

The terms “polynucleotide” or “oligonucleotide” or “nucleic acid” can be used interchangeably and includes single-stranded or double-stranded polymers of nucleotide monomers, including 2′-deoxyribonucleotides (DNA) and ribonucleotides (RNA) linked by internucleotide phosphodiester bond linkages, or internucleotide analogs, and associated counter ions, for example, H+, NH4+, trialkylammonium, Mg2+, Na+ and the like. A polynucleotide can be composed entirely of deoxyribonucleotides, entirely of ribonucleotides, or chimeric mixtures thereof. Polynucleotides can be comprised of nucleobase and sugar analogs. Polynucleotides typically range in size from a few monomeric units, for example, 5-40 when they are frequently referred to in the art as oligonucleotides, to several thousands of monomeric nucleotide units. Unless denoted otherwise, whenever a polynucleotide sequence is represented, it will be understood that the nucleotides are in 5′ to 3′ order from left to right and that “A” denotes deoxyadenosine, “C” denotes deoxycytidine, “G” denotes deoxyguanosine, and “T” denotes thymidine, unless otherwise noted. A labeled polynucleotide can comprise modification at the 5′terminus, 3′terminus, a nucleobase, an internucleotide linkage, a sugar, amino, sulfide, hydroxyl, or carboxyl. See, for example, U.S. Pat. No. 6,316,610 B2 to Lee et al. which is incorporated herein by reference. Similarly, other modifications can be made at the indicated sites as deemed appropriate.

### DETAILED DESCRIPTION OF EMBODIMENTS

For simplicity and illustrative purposes, the principles of the present invention are described by referring mainly to exemplary embodiments thereof. However, one of ordinary skill in the art would readily recognize that the same principles are equally applicable to, and can be implemented in, all types of detection systems, and that any such variations do not depart from the true spirit and scope of the present invention. Moreover, in the following detailed description, references are made to the accompanying figures, which illustrate specific embodiments. Electrical, mechanical, logical and structural changes may be made to the embodiments without departing from the spirit and scope of the present invention. The following detailed description is, therefore, not to be taken in a limiting sense and the scope of the present invention is defined by the appended claims and their equivalents.

Some embodiments generally relate to systems, apparatus, and methods for generating a high energy field through the use of surface plasmons, where the surface plasmons are located on the periphery of a resonant cavity. More particularly, the resonant cavity may be implemented with metallic nanoparticles. For example, nanorice can be placed in an analyte solution and facilitate detection of events in a confined space. An excitation light can create plasmons, i.e., the localized high energy field on the surface of the nanorice., which then can be applied to the analyte. Other examples of metallic nanoparticles can be nanorods, nanorings, nanocubes, nanoshells and nanocrescents. The nanoparticles can be varied in size and aspect which allows the nanoparticles to be tuned to vary the absorption spectra of the nanoparticle and the energy of the generated plasmon. The embodiments that create a localized plasmon resonance, may then be used in applications such as single-molecule detection and fluorescent correlation spectroscopy (“FCS”). Other applications include single molecule sequencing and multiple molecule sequencing.

Another embodiment generally relates to a sub-wavelength hole array of appropriate thickness and material such that plasmon resonance is generated on the peripheral surface surrounding one of the holes in the hole array, thus, enhancing the energy available as well as placing it in a small volume. The excitation light is directed to the surface of the hole array. Some of the light is reflected or may enter a hole in the hole array, but the majority of the energy is coupled in from light that strikes the surface periphery of the hole. The coupling of the light generates a plasmon resonance in the hole, through the hole, and/or at a planar surface above the hole. Similar embodiments may include appropriate dielectric materials such that the plasmon resonance is maintained.

Yet another embodiment relates generally to a photonics crystal used as a sub-wavelength waveguide. More particularly, a similar sub-wavelength hole array may hold the target analyte. The photonics crystal waveguide directs the excitation light, allowing recycling of expensive laser light.

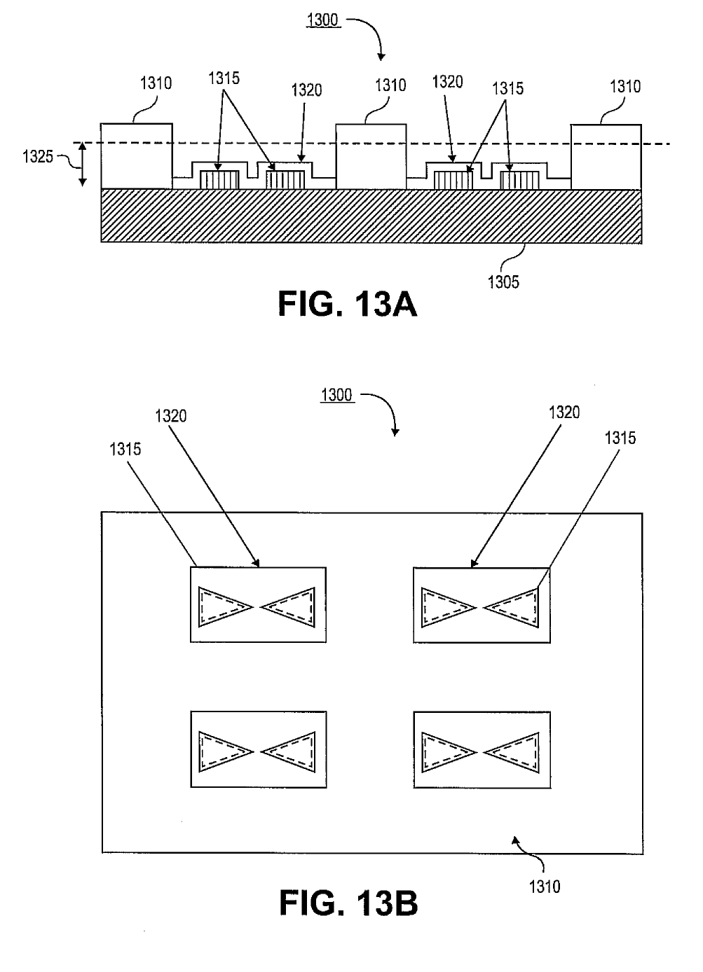

Yet another embodiment pertains generally to nano-antennas to focus plasmon energy to a localized spot. For example, a circular nano-antenna can be fabricated. One property of circular nano-antennas is that they focus energy to the center, thus increasing plasmon intensity in a localized area. Another example of a nano-antenna is a bow tie nano-antenna. Nano-antennas can also be used as a receiver. As such, they can be used to quench a molecule as well as to collect emissions. All of these metallic structures quench fluorescence if the fluorophore is close enough. To prevent undesired quenching the fluorophore can be spaced off the metal using a thin (approx 5-20 nm) dielectric layer. Such a layer can be made of glass, plastic or a chemical coating such as PEG. The thickness should be sufficient to space off a fluorophore so that it is not completely quenched, but not so far that it is spaced outside of the volume of the concentrated plasmons. As shown in FIG. 13A and 13B one could selectively coat the surface providing a greater thickness in the area away from the nanoantenna. This can minimize background by placing a low fluorescent material in the steep part of the exponential decay of the evanescent wave excitation zone. The evanescent wave zone could be created by SPR or by TIRF as taught in U.S. Provisional Application, 60/800,440 filed on May 16, 2006, which is hereby incorporated by reference in its entirety.

Embodiments of the invention are generally directed to creating a high energy field in a small volume, i.e. sub-wavelength dimensions. One embodiment utilizes nanoparticles. It is known that solid metal nanoparticles (i.e. solid, single metal spheres of uniform composition and nanometer dimensions) possess unique optical properties. In particular, metal nanoparticles (especially the coinage metals) display a pronounced optical resonance. This so-called plasmon resonance is due to the collective coupling of the conduction electrons in the metal sphere to the incident electromagnetic field. This resonance can be dominated by absorption or scattering depending on the radius of the nanoparticle with respect to the wavelength of the incident electromagnetic radiation. Associated with this plasmon resonance is a strong local field enhancement on the surface of the metal nanoparticle.

However, a serious practical limitation to realizing many applications of solid metal nanoparticles is the inability to position the plasmon resonance at technologically important wavelengths. For example, solid gold nanoparticles of 10 nm in diameter have a plasmon resonance centered at 520 nm. This plasmon resonance cannot be controllably shifted by more than approximately 30 nanometers by varying the particle diameter or the specific embedding medium.

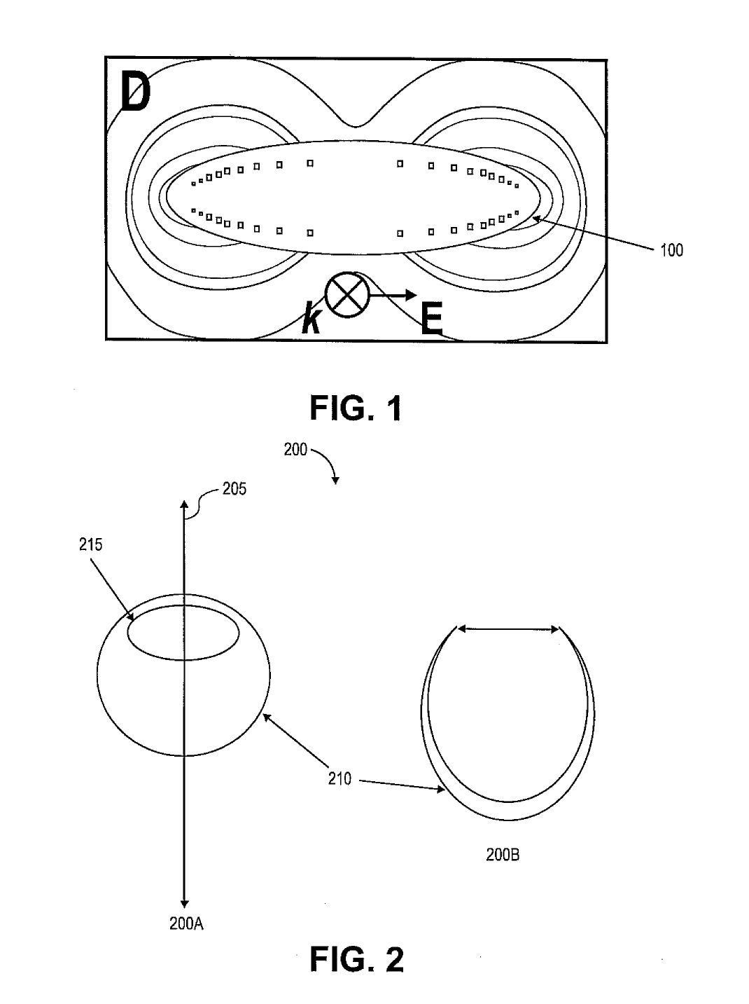

Accordingly, composite nanoparticles have been fabricated to that allow the plasmon resonance centered around a desired wavelength. FIG. 1 illustrates exemplary nanorice, a type of nanoparticle, in accordance with an embodiment of the present invention.

As shown in FIG. 1, nanorice 100 is shaped similar to a grain of rice. Nanorice 100 can be made out of non-conducting iron oxide called hematite that's covered with gold. The thickness of the shell, length of the nanorice, and width of the core can be manipulated to generate a specific frequency of plasmon resonance. The method of fabrication for nanorice 100 is described in Nanorice: A Hybrid Plasmonic Nanostructure, Nano Let., 6(4), 827-837, 2006 Hui Wang et al., which is incorporated by reference in its entirety.

In some embodiments, an excitation light source (not shown) may be directed at the nanorice 100. The excitation light source can be a laser, laser diode, a light-emitting diode (LED), an ultra-violet bulb, and/or a white light source. Plasmons are collective oscillations of free electrons at optical frequencies that travel across the metal surface of nanorice 100. Plasmons on the surface of nanorice 100 can convert light into electrical energy when the frequency of the light resonates with the frequency of the plasmon's oscillation. This resonant effect can create high intensity local electrical fields that radiate around the particle. Accordingly, FlG. 1 also illustrates the strong energy fields created by plasmon resonance near the ends of a grain of nanorice 100. The unique shape of the nanorice allows for stronger fields than those previously measured in rod-shaped and spherical particles.

Accordingly, the nanorice 100, may be positioned within an analyte. Excitation light can be directed at the nanoparticle to generate plasmons in a small volume. This method of generating plasmons has a side benefit that bleaching does not occur as quickly as in conventional methodologies. The nanoparticle causes the fluorescence lifetime of the fluorophore to decrease, which increases the fluorescence photon emission rate and the total number of emitted photons before bleaching.

In other embodiments, other nanostructuctures can be use in lieu of the nanorice. For example, nanorods, nanorings, nanocubes and nanoshells can be used, depending on the user-requirement. Each of the nanostructures exhibit their own resonant wavelength, intensity of field, number of field generated, etc.

FIG. 2 illustrates two view of an exemplary nanocrescent 200. In view 200A, represents a three dimension view of the nano-crescent 200 and 200B represents a profile view of the nano-cresecent 200 as bisected by axis 205. The nanocrescent 200 can comprise a metal shell 210 with a circular section removed from one side. The metal shell 210 maybe implemented with gold, iron, silver, and or combinations thereof. During the fabrication of the nano-crescent 200, metal is deposited over most of a dielectric core. The dielectric core is then removed.

After the dielectric core is removed, the nano-crescent 200 may be a spheroid object with a circular area 215 removed from the shell. In the view 200B, a cross section of the nano-crescent 200 appears to come to sharp points. However, from the view in 200A, the sharp points are actually part of a circle.

In accordance with various embodiments, excitation light can be directed at the circular area 215 where surface plasmons on the periphery of the circular area 215 can couple with the excitation light and create a resonant field. In essence the nano-crescent 200 can be functioning as a resonance structure, which then can be applied to applications such as single-molecule sequencing, hybridization or other applications directed at detecting small particles with a reduced background clutter as compared to conventional systems. Moreover, the angle of the excitation light or the orientation of the nano-crescent 200 will affect the number of plasmons being generated as well as efficiency and location of the plasmons.

The nanocrescent 200 can be implemented as described in Magnetic Nanocrescents As Controllable Surface-Enhanced Raman Scattering Nanoprobes For Biomolecular Imaging, Liu et a., Advanced Materials 2005, 17, 2131-2134 and Advanced Materials 2005, 17, 2683-2688 Luke P. Lee at al. UC Berkeley, which are hereby incorporated by reference in their entirety.

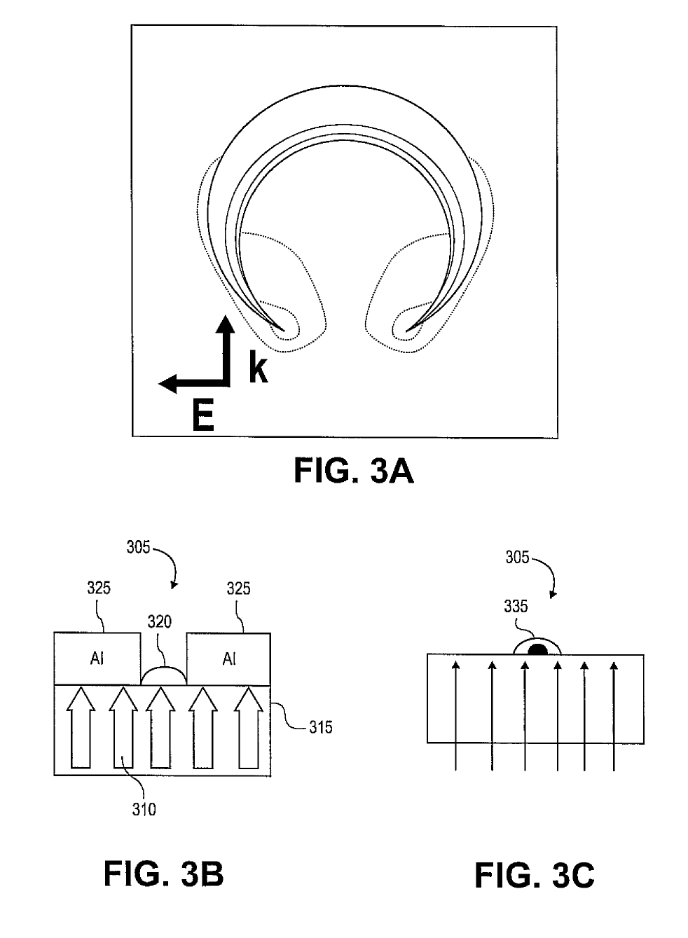

FIG. 3A illustrates intensity image of a nanocrescent. As seen in FIG. 3, the field is greatest where the metal forms a circle.

FIG. 3B illustrates an apparatus 305 for creating a small excitation volume. As shown in FIG. 3B, the apparatus 305 receives energy 310 from an excitation source through a substrate 315. The energy generates an evanescent zone (not shown) which covers an analyte 320. The small excitation zone is maintained around the analyte 320 by a blocking material 325, which blocks the excitation light. This apparatus 305 can require the use of high power lasers and can generate a considerable amount of background and associated noise.

FIG. 3C illustrates a generalized embodiment with the use of the resonant structures described with respect to FIGS. 1-3A. More particularly, the resonant apparatus 330 can be configured to create a small excitation volume 335 by strongly enhancing laser excitation in the vicinity of the enhancing resonant structure not shown. The FER can show an improvement over apparatus 305. Moreover, the power requirement for the excitation source is lessened, reducing the amount of background and associated noise.

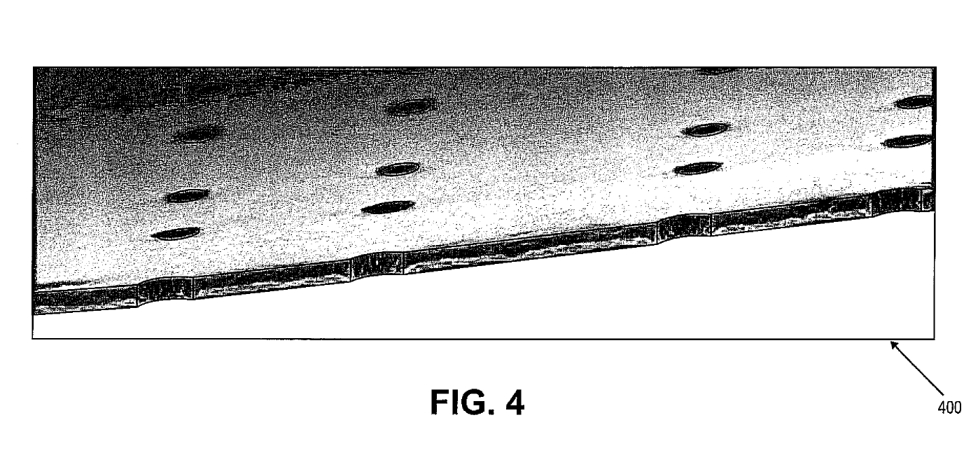

FIG. 4 illustrates a sub-wavelength hole array 400 in accordance with yet another embodiment. As shown in FIG. 4, the hole array 400 can be fabricated of thickness and material as known to those skilled in the art such that plasmon resonance can be generated through a hole in the hole array 400. An example is a plasmon resonance at 488 nm with a hole diameter of 60 nm. The excitation light source may be an Argon-Ion laser at 488 nm.

In some embodiments, nanoparticles such as a nanorice or nanocrescent, or other nano-antennas such as a bow-tie may also be placed on the periphery of the holes, or at resonant points between the holes to further enhance the plasmon resonance output within the array 400. This may be done to further concentrate or enhance the plasmons into a small area. The nanoparticles or nano-antennas could also be placed on a dielectric material which fills or partly fills the holes, and could also be placed inside the holes on a dielectric which does not fill or partly fill the hole, for the purpose of further concentrating the plasmons.

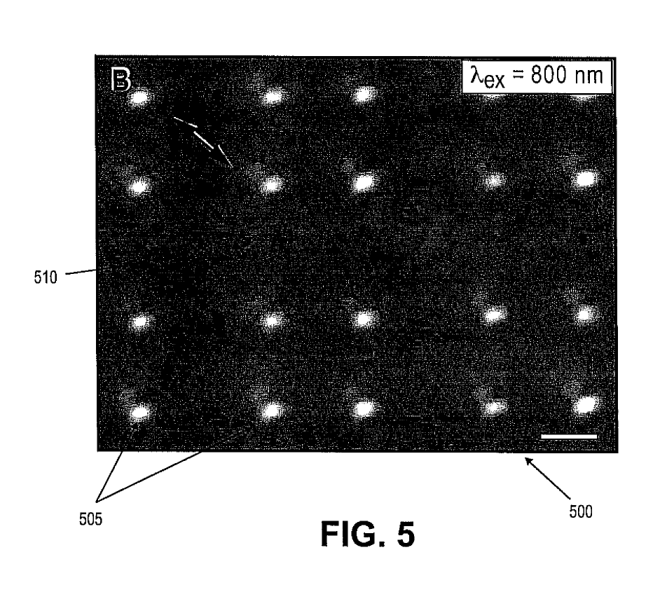

FIG. 5 illustrates a near field scanning microscope image 500 of an energy pattern of the array 400. As shown in FIG. 5, the image 500 shows the holes 505 as bright lights while the background 510 as substantially dark.

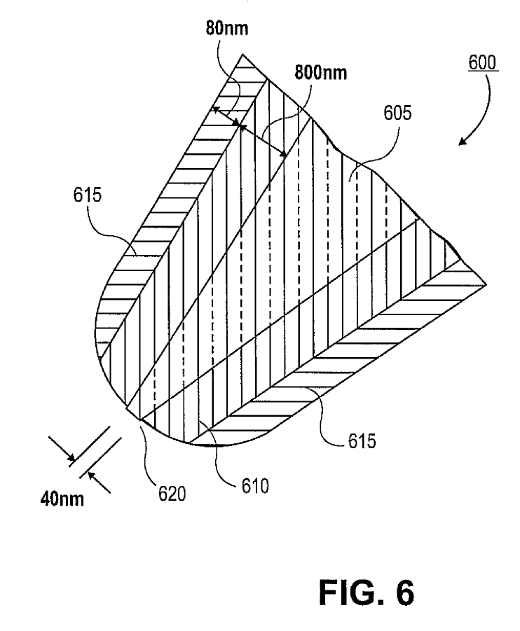

FIG. 6 illustrates a blunt tip optical fiber 600 in accordance with yet another embodiment. The construction of this tip is described in U.S. Pat. No. 5,812,724, which is hereby incorporated by reference. As shown in FIG. 6, the blunt tip optical fiber 600 has, on one end of the optical fiber 600, a tip 605 that is even with the cladding (not shown). In other embodiments the tip 605 may be rise above the cladding but be blunted as shown in FIG. 6. This optical fiber 600 has a coating layer 610 on the surface of the tip 605 and a corrosion-resistant coating layer 615 on an area of the surface of the light-shielding coating layer 610 other than the foremost part of the surface of the light-shielding layer 610. The foremost part of the tip 605 has an aperture 620 which is exposed from the light-shielding coating layer 610 and the corrosion-resistant coating layer 615. The light-shielding coating layer 610 is formed of, for example, aluminum, and has a thickness on the order of 800 nm. The aperture 620 has a diameter of, for example, 40 nm.

In various embodiments, the blunt tip optical fiber 600 can be positioned outside a target analyte containing nanoparticles. An evanescent wave from plasmons resulting from the excitation light can then be passed into the target analyte. In other embodiments, the blunt tip optical fiber 600 may be replaced with a optical tip with a protruding tip as well as configured in array of tapered fiber optics.

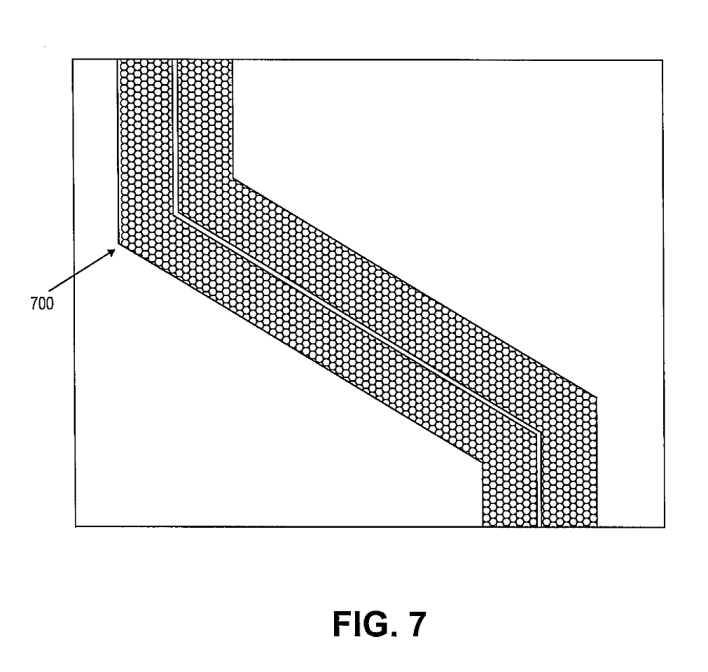

FIG. 7 illustrates a planar photonic waveguide structure 700 in accordance with yet another embodiment. As shown in FIG. 7, the planar photonic waveguide structure 700 can be implemented as described in Maier et al., Proceedings of SPIE Vol. 8410 and in Design and Fabrication of Photonics Crystal waveguides, Loncar et al., Journal of Lightwave Technology Vol. 18, No. 10, which are hereby incorporated by reference in their entirety. The planar photonic waveguide structure 700 can be configured as a line source instead of a point light source for some of the previously described embodiments. For example, the nanoparticles or the hole arrays.

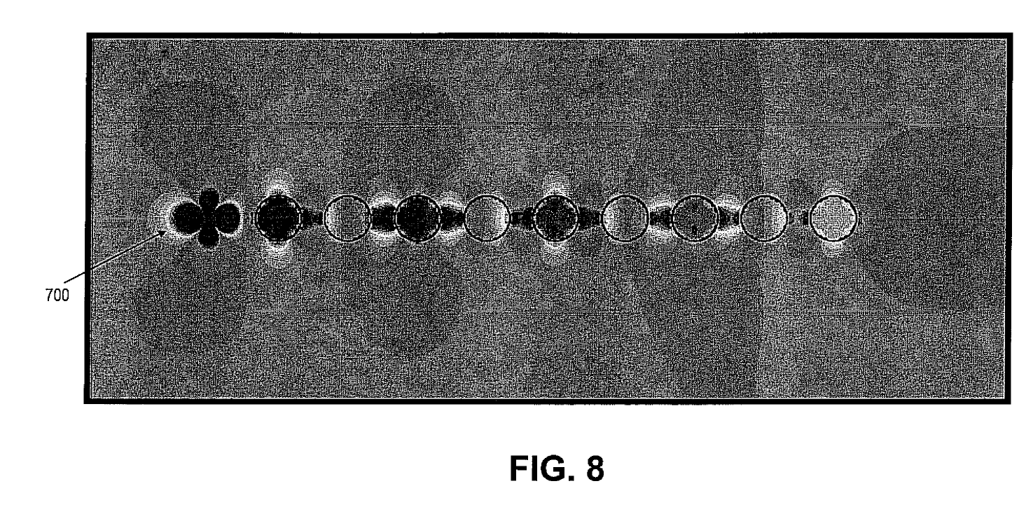

FIG. 8 shows an intensity of profile of a planar photonic waveguide structure. The waveguide structure can be implemented by using a photo-resist to pattern openings within strips in the opposite axis. In some embodiments, holes can be used as well. Photoactivation of the attachment would occur as a result at the intersection of the missing photo-resist and the plasmon waveguide.

Moreover, a nano-antenna, nanoparticles, colloidal particle or a quantum dot may be placed close to the plasmon waveguide, such as waveguide 700, and thus permitting direct coupling between the waveguide and the nanoparticle. The photonic crystal structure permits bending of the light around corners, and thus permitting the light to be rastered back and forth over a field of view of a far field microscope. This enables the light energy to be recycled as it is directed over the field of view. In addition, the energy is localized to the path of the waveguide reducing unwanted background. Multiple waveguides can be used to efficiently cover a large area.

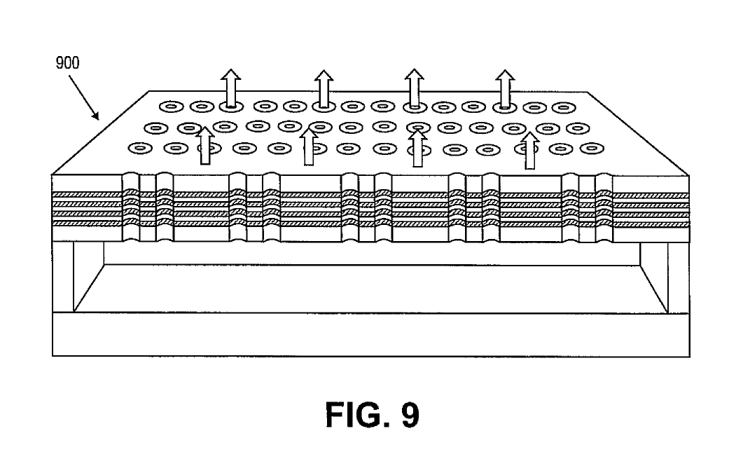

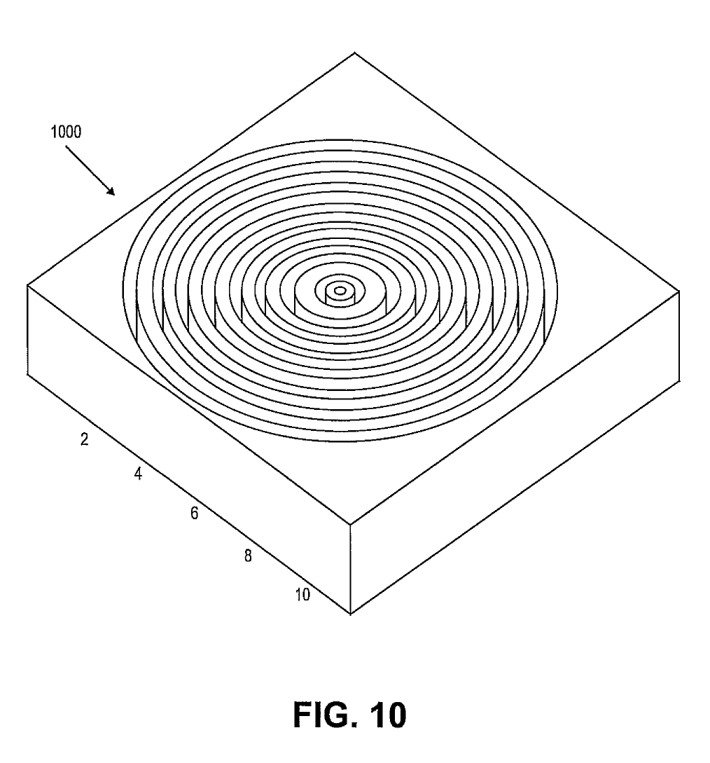

In other embodiments, a two-dimensional photonic crystal can be used to create an appropriate two-dimensional intensity profile, which is described in Photonic Crystal Nano-cavity Arrays, Altug et al., IEEE LEOS Newsletter, April 2006 and hereby incorporated by reference in its entirety. FIG. 9 illustrates an embodiment of the two dimensional photonic crystal 900

FIG. 10 illustrates an exemplary nano-antenna 1000 in accordance with an embodiment. As shown in FIG. 100, the nano-antenna 1000 is a circular nano-antenna configured to focus plasmon energy to a localized spot. The nano-antenna 1000 can be positioned over a dielectric material. As excitation light is directed on the nano-antenna 1000, the nano-antenna 1000 directs plasmons to the center of the antenna 1000.

This type of circular nano-antenna 1000 may be implemented as a set of concentric circular first material swaths alternative disposed with circular swaths of a second material over a substrate material. In the embodiment shown in FIG. 10, the center of the circular nano-antenna 1000 is implemented with the first material and the second material being absent. Other embodiments of the circular nano-antenna 1000 may reverse the order of materials, where center is implemented with the second material alternating with the first material. Other embodiments can include circular nano-antenna implemented with a second material that would block any excitation light, thus reducing background and associated noise.

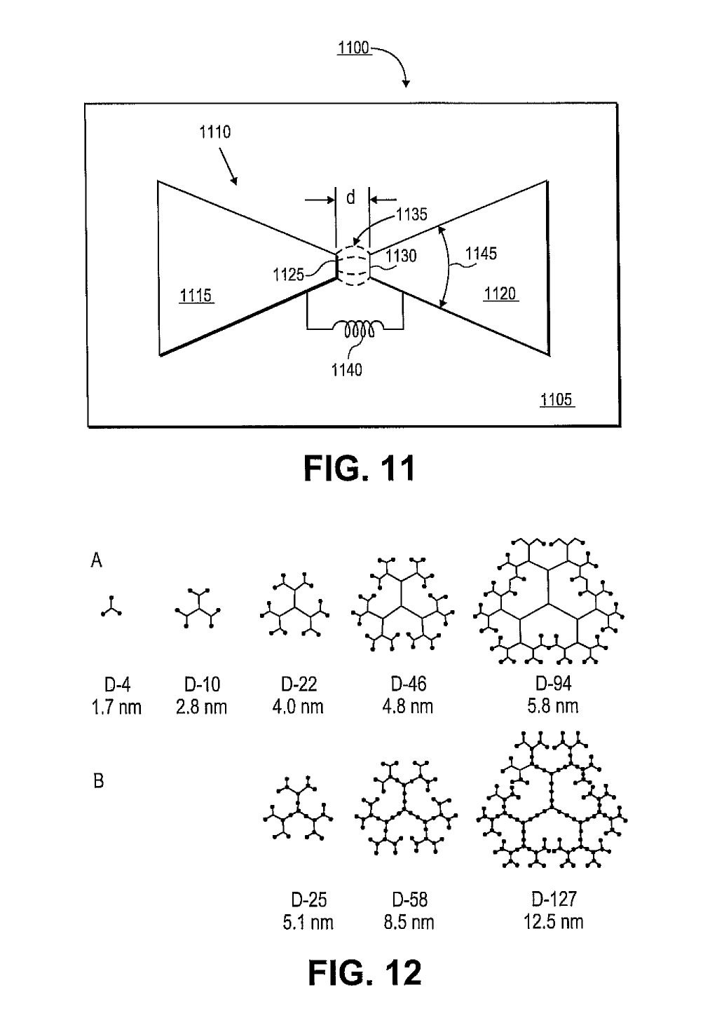

FIG. 11 illustrates a bow-tie nano-antenna 1100. As shown in FIG. 11, the bow tie nano-antenna 1100 can comprise an electromagnetically transparent substrate 1105 upon which an antenna structure 1110 is supported. The antenna structure 1110 comprises a bowtie antenna including conductive arms 1115 and 1120, respectively. At terminations 1125 and 1130, conductive arms 1115 and 1120 are separated by a gap 1135 having a transverse dimension d. In essence, conductive arms 1115 and 1120 form a dipole-like antenna. Other antenna structures will work with the invention, such as log-periodic, spiral and slot antennas. More detailed description of the bow-tie antenna can be found in U.S. Pat. No. 5,696,372, which is hereby incorporated by reference in its entirety.

Gap 1135 forms an emission “region” between terminations 1125 and 1130 of conductive arms 1115 and 1120. The transverse dimension “d” between terminations 1125 and 1130 is small in relation to the wavelength of the incident electromagnetic energy.

It is preferred that the incident energy have a wavelength in the optical range, however, it is to be understood that the invention is equally applicable to non-optical wavelength applications.

From a review of FIG. 11 it can be seen that terminations 1125 and 1130, separated by gap 1135, constitute a capacitance. In order to more efficiently impedance match the capacitance of gap 1135 to the antenna structure, and improve the coupling of energy thereunto, it is preferred to connect an inductor 1140 in parallel with region 1135 to create a tuned circuit. The essential idea is to match the antenna impedance to the radiation resistance of the dipole radiator formed at gap 1135. The angle 1145 of the conductive arms 1115 and 1120 may be implemented at a variety of angles dependent on the desired frequency.

FIG. 12 illustrates a selection of nano-fractal antenna patterns. The selection of the type of fractal type can be dependent on user-desired performance characteristics. In other embodiments, the fractal nano-antenna can also be a linear dipole.

In yet other embodiments, the nano-antennas 1000, 1100, 1200 can also be used as-a receiver. As such, these antennas can be used to quench a molecule as well as collect emission.

FIGS. 13A-B depict another embodiment that relates generally to a use of a coating over a bulk substrate or dielectric material, as well as over a nano-antenna. As shown in FIGS. 13A-B, a coated enhancement structure 1300, shown in profile view, includes a substrate 1305. The substrate 1305 may be implemented to generate an evanescent zone 1325, or optionally using a metal layer (not shown) in order to use plasmon resonance instead of TIRF. A thick coating 1310 may be applied to the substrate 1305 in such a way that an area is left free of the thick coating 1310. The thick coating 1310 can be implemented with a dielectric material greater than the evanescent zone 1325 that included most of the evanescent wave energy.

In the open areas, bow tie antennas 1315 can be formed. In other embodiments, other fractal nano-antennas may be used. In yet other embodiments, the previously described resonant structures can be placed in the open areas. A thin coating 1320 may be deposited in the open areas covering the resonant structure. Alternatively, a thin coating may be placed over the entire surface, and a thicker coating may optionally be added later. The thin coating 1320 may the same or another dielectric material with a thickness selected to optimize the balance between quenching and excitation.

The thin coating 1320 can be configured to stand off a fluorophore to prevent quenching, being of a thickness of 5 to 20 nm. The thick coating 1310 can be made out of a material of appropriate lower refractive index (relative to the substrate) that blocks fluorophore access to the volume of the highest intensity of TRF (total internal reflection fluorescence). Accordingly, the background and associated noise is reduced but not eliminated.

For all the disclosed embodiments, a target DNA, a primer or an enzyme can be attached to the surface in the area of highest energy intensity. One method of creating this attachment can utilize a photo-activated attachment such as photo-activated biotin. At low intensity light levels, the molecules would be preferentially attached at the point of highest energy on the structure. The excitation or emission could use the disclosed methods either individually or in combination with other conventional methodologies such as far field microscopy, TIRF, plasmon resonance or other methods of coupling to provide energy to the structures. Use of TIRF or plasmon resonance minimizes the excitation to a very thin layer reducing unwanted background. The depth of penetration of the evanescent wave resulting from TIRF excitation is a function of the angle incidence, where the penetration is greatest at the critical angle, and diminishes as the angle between the substrate and the excitation light decreases. Thus, to minimize the depth of penetration, and thus the volume of solution which is excited by the evanescent wave, it is preferable to minimize the angle. For example, this can be accomplished by using a high NA TIRF objective, utilizing a laser brought in at the extreme edge of the objective.

The device may be used for single molecule fluorescence. The device may be used to create two-photon emission from dyes using the wavelength of the antenna/nanoparticle instead of the excitation wavelength. Two-photon emission requires two photons to excite a molecule prior to the emission of a photon. With two-photon emission, the generated fluorescence is at a wavelength lower than the excitation, permitting easy filtering of background fluorescence of the substrate, optical elements and other nonspecific fluorescence. Furthermore, the probability that two-photon emission will occur is a function of the excitation power square, thus, if a device has an optical enhancement of 100, a fluorophore in an resonant enhancement zone is actually 10,000 times more likely to be excited than a fluorophore which is not in resonant enhancement zone, greatly reducing background from nearby fluorophores. As such, they could be used for DNA sequencing but also for many other types of applications where it is desired that small volumes be excited.

While the invention has been described with reference to the exemplary embodiments thereof those skilled in the art will be able to make various modifications to the described embodiments without departing from the true spirit and scope. The terms and descriptions used herein are set forth by way of illustration only and are not meant as limitations. In particular, although the method has been described by examples, the steps of the method may be performed in a different order than illustrated or simultaneously. Those skilled in the art will recognize that these and other variations are possible within the spirit and scope as defined in the following claims and their equivalents.

What is claimed is:

1. A method for analysis of a nucleic acid, the method comprising:
providing for a resonant structure configured to couple with one or more fluorescently labeled nucleic acids;
directing an excitation light from a source on the resonant structure; and
generating plasmons on the surface of the resonant structure wherein the analyte is fixed at a point of energy concentration of the resonant structure.

2. A method for analysis of an analyte, the method comprising:
providing for a resonant structure coupled with an analyte;
directing an excitation light from a source on the resonant structure; and
generating plasmons on the surface of the resonant structure, wherein the analyte is complexed with a molecule fixed at a point of energy concentration of the resonant structure through a photoactivatable linker.

3. The method of claim 2 where the plasmons are used in single molecule sequencing.

4. The method of claim 2 where the plasmons are used in fluorescent correlation spectroscopy.

5. The method of claim 2, wherein the resonant structure is a nano-particle.

6. The method of claim 5, wherein the nanoparticle is one of nanorice, nanorods, nanorings, nanocubes, nanoshells, and nanocrescents.

7. The method of claim 6, wherein the plasmons are generated on the periphery of the nanocrescent.

8. The-method of claim 2, wherein the resonant structure is an array of holes.

9. The method of claim 8, wherein the plasmons are generated on surface of a hole in the array of holes, above the array of holes and through the holes.

10. The method of claim 2, wherein the excitation light source is a blunt fiber optic tip.

11. The method of claim 10, wherein the excitation light source is positioned outside the analyte.

12. The method of claim 10, wherein the excitation light source is an array of fiber optic tips.

13. The method of claim 2, wherein the resonant structure includes a photonic sub-wavelength waveguide.

14. The method of claim 2, wherein the resonant structure includes a two-dimensional photonic crystal.

15. The method of claim 2, wherein the resonant structure is a nano-antenna.

16. The method of claim 2, wherein the resonant structure is a bow-tie nano-antenna.

17. The method of claim 16, further comprising providing for a coating on the bow-tie antenna, wherein the coating is configured to be of appropriate thickness to substantially prevent quenching.

18. The method of claim 1, further comprising providing for a photo-activatable attachment at the point of energy concentration of the resonant structure.

19. The method of claim 18, wherein the photo-activatable attachment is part of single molecule sequencing.

20. A plasmonic structure, comprising:
a nano-antenna implemented with a metal material and configured to generate an enhancement zone; and
a blocking layer deposited adjacent to a portion of the nano-antenna, wherein the blocking layer is configured to substantially reduce the excitation of fluorophores outside of the enhancement zone.

21. The plasmonic structure of claim 20, wherein the blocking layer is implemented with a dielectric.

22. The plasmonic structure of claim 20, further comprises a metal layer wherein the evanescent wave excitation zone is generated by SPR through the metal layer.

23. The plasmonic structure of claim 20, wherein the evanescent wave excitation zone is generated by TIRE.
